(19) United States
(12) Patent Application Publication
(10) Pub. No.: US 2007/0298106 A1
(43) Pub. Date: Dec. 27, 2007

(54) Sustained Release Formulation Containing Selective Serotonin Reuptake Inhibitor and Method for the Preparation Thereof

(75) Inventors: Jin Woo Park, Seoul (KR); Young Hee Shin, Uiwang-si (KR); Jung Ju Kim, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: Amorepacific Corporation, Seoul (KR)

(21) Appl. No.: 11/719,971

(22) PCT Filed: Nov. 30, 2005
(86) PCT No.: PCT/KR05/04060

§ 371 Date: May 23, 2007

(30) Foreign Application Priority Data
Nov. 30, 2004 (KR) 10-2004-0099426

(51) Int. Cl. A61K 9/22; A61K 9/28; A61P 25/24

(52) U.S. Cl. 424/470; 424/474; 514/321

(57) ABSTRACT

A sustained release formulation of a selective serotonin reuptake inhibitor (SSRI), comprising the SSRI as an active ingredient; a matrix comprising a water-soluble and erodible polymer, a hydrogel for shape sustenance and a pharmaceutically acceptable additive, the SSRI being embedded in the matrix, is capable of maintaining a constant drug level in the blood owing to the fact that the release of the SSRI follows zero order kinetics without initial burst release and such a release pattern is little affected by external factors such as gastrointestinal movements.

### FIELD OF THE INVENTION

The present invention relates to a sustained release formulation of selective serotonin reuptake inhibitor and a method for the preparation thereof.

### BACKGROUND OF THE INVENTION

A selective serotonin reuptake inhibitor (SSRI) such as paroxetine is useful for treating or preventing depression through long-term administration thereof. A sustained release formulation of a drug is preferably used in such long-term administration, it can maintain an effective, constant in vivo drug level over a long period. In particular, sustained release formulations are preferred for a drug which tends to adversely stimulate the stomach and intestines.

A sustained release formulation, which maintains a constant rate of drug release following zero order kinetics, may be achieved by either osmotic release control or matrix-based release control.

A typical osmotic formulation is capable of releasing a drug at a constant rate driven by the osmotic pressure difference. However, it is disadvantageous in that an expensive and complicated structure having a multi-layered inner core and multi-coatings formed thereon is required. In particular, an osmotic formulation for a water insoluble drug conventionally contains a solubilizer for drug release, the solubilizer often causing irregular release of the drug.

A matrix-based formulation typically releases a drug via diffusion through a matrix in which the drug is embedded. However, a constant release rate of the drug cannot be obtained due to the gradual reduction of the drug concentration gradient in the matrix and the gradual increase in the diffusion distance. Accordingly, there have been attempts to control the release rate of a drug by using a hydrophilic polymer having erodible and swelling properties as a matrix component, morphologically controlling the matrix area.

A hydrophilic polymer-based matrix consists of swelling, diffusion and erosion layers. A constant release rate of a drug may be achieved by equalizing the drug mobilities in the diffusion and erosion layers. However, such a balance of the mobilities is difficult to attain because diffusion tends to prevail over erosion in such a matrix, and to reduce the mobility of the diffusion layer an excessive amount of a hydrophilic polymers must be used (Carmen F. R. et al., Handbook of Pharmaceutical Controlled release Technology, 1-30, 2000).

In order to reduce the use of the polymer, EP Patent No. 0 654 263 A1 discloses a method for preparing controlled-release particles comprising mechanically mixing a drug such as fluoroxetin or paroxetine and a hydrophobic and/or hydrophilic fusible carrier using a high speed mixture; and PCT WO 96/31197 discloses a method for preparing a controlled-release formulation comprising melt-mixing a drug having a low melting point such as paroxetine hydrochloride and an additive for sustained release of the drug to produce a homogeneous mixture, filling the mixture in a capsule and cooling. However, these methods comprising heating and cooling steps suffer from the problem of drug degeneration.

Further, U.S. Pat. No. 6,548,084 B2 discloses a method for reducing the nausea and vomiting side effects associated with an SSRI such as fluvoxamine, sertraline, fluoroxetin and paroxetine by providing the formulation of an enteric coated controlled-release bilayer tablet comprising a drug layer and a supporting layer thereof, the supporting layer partially covering the surface of the drug layer to prevent the burst release of the drug. However, the preparation of such a bilayered tablet is very complicated and the drug release pattern may be adversely affected by external influences.

Also, most of the conventional hydrophilic polymers-based matrices have the common problem of easy destruction by the contractive movements of the gastrointestinal organs (Masaharu K. et al., International Journal of Pharmaceutics, 237, 2002).

### SUMMARY OF THE INVENTION

Accordingly, it is an object of the present invention to provide a sustained release formulation which can maintain a constant release rate of a selective serotonin reuptake inhibitor for a long period which is little affected by the degree of gastrointestinal motility.

It is another object of present invention to provide a method for preparing the sustained release formulation under a mild condition.

In accordance with one aspect of the present invention, there is provided a sustained release formulation of a selective serotonin reuptake inhibitor (SSRI), comprising the SSRI as an active ingredient and a matrix comprising a water-soluble and erodible polymer, a hydrogel for shape sustenance and a pharmaceutically acceptable additive, wherein the SSRI is embedded in an efficient amount in the matrix.

### BRIEF DESCRIPTION OF DRAWINGS

The above and other objects and features of the present invention will become apparent from the following description of the invention taken in conjunction with the following accompanying drawings, which respectively show:

FIG. 1: in vitro release profiles of the sustained release formulations prepared in Examples 10 and 14 of the present invention; and

FIG. 2: in vitro release profiles of the sustained release formulation prepared in Example 20 of the present invention.

### DETAILED DESCRIPTION OF THE INVENTION

The sustained release formulation of the prevent invention comprises a selective serotonin reuptake inhibitor (SSRI) as an active ingredient, and a single layered matrix comprising a water-soluble and erodible polymer, a hydrogel for shape sustenance and a pharmaceutically acceptable additive.

The inventive sustained release formulation can release the SSRI at a constant rate following zero order kinetics owing to the use of the hydrogel for shape sustenance. The hydrogel plays a role in controlling the surface erosion of the matrix to prevent drug destruction due to external factors such as gastrointestinal movement and foods. Also, the release rate is represented by zero order release rate with a correlation coefficient of 0.8 to 1.0.

In the present invention, the weight ratio of polymer:hydrogel may ranges from 1:0.1 to 1:8, preferably from 1:0.1 to 1:3 and more preferably 1:0.1 to 1:2.

Each ingredient of the inventive formulation is described in detail as follows.

The term “a” or “an” used herein do not denote a limitation of quantity, but rather denote the presence of at least one of the referenced item.

The term “viscosity” used herein means the value obtained for an aqueous solution (1 to 2 wt %) of a material at 20° C.

The pharmacologically active ingredient (drug) of the inventive formulation is an SSRI selected from the group consisting of paroxetine, sertraline, fluoxetine, fluvoxamine and pharmaceutically acceptable salts thereof, and has a water-solubility of 10 mg/ml or less.

The drug is used in an amount of 0.5 to 80 wt %, preferably 1 to 30 wt % based on the total weight of the formulation. When the amount of the drug is less than 0.5 wt %, a homogeneous dispersion of the drug cannot be obtained, and when higher than 80 wt %, a desired sustained release effect cannot be achieved.

The drug release rate of the inventive formulation may vary depending on the type, viscosity, particle size and molecular weight of the water-soluble and erodible polymer. The polymer preferably has a pH-independent viscosity ranging from 3 to 400 centipoise (cps). Exemplary water-soluble and erodible polymers include hydroxyalkyl cellulose, hydroxypropyl alkylcellulose, polyethylene oxide, propylene glycol alginate, polyvinylpyrrolidone, polyvinylalcohol, sodium carboxymethyl cellulose and a mixture thereof. Among these, hydroxyethyl cellulose, and hydroxypropyl methylcellulose which has a methoxyl group content of 27 to 30% and a hydroxypropoxyl group content of 4 to 12%, are particularly preferred.

The water-soluble and erodible polymer is used in an amount of 5 to 40 wt % based on the total weight of the formulation. When the amount of the polymer is less than 5 wt %, it becomes difficult to control the drug release rate by the erosion of the polymer, and when higher than 40 wt %, the formulation becomes oversized.

The hydrogel for shape sustenance used in the present invention may be a water-insoluble polymer, a water-soluble polymer having a solubility lower than that of the water-soluble, erodible polymer and a viscosity of 100 to 200,000 cps, or an enteric polymer whose solubility changes according to pH, in order to minimize the drug loss due to its pre-mature release in the stomach or gastrointestinal movements.

Exemplary hydrogels include hydroxyalkyl cellulose, hydroxypropyl alkylcellulose, sodium alginate, xanthan gum, locust bean gum, cellulose gum, ethyl cellulose, ammonio methacylate copolymer, anionic copolymer of methacrylic acid and methyl or ethyl methacylate, hydroxypropyl methylcellulose acetate succinate, hydroxypropyl methylcellulose phthalate, carbopol and a mixture thereof. Among these, hydroxypropyl cellulose, and hydroxypropyl methylcellulose which has a methoxyl group content of 19 to 24% and a hydroxypropoxyl group content of 4 to 12% are preferred.

The hydrogel for shape sustenance is used in an amount of 5 to 40 wt % based on the total weight of the formulation. When the amount of the hydrogel is less than 5 wt %, the degree of shape sustenance becomes low, and when higher than 40 wt %, the release rate of the drug becomes excessively slow.

The sustained release formulation of the present invention may further comprise pharmaceutically acceptable additives such as diluents, binders and lubricants. Exemplary such diluents include lactose, dextrin, starch, microcrystalline cellulose, calcium hydrogen phosphate, anhydrous calcium hydrogen phosphate, calcium carbonate, sugars, cross-linked sodium carboxymethyl cellulose, cross-linked polyvinylpyrrolidone, mannitol, saccharose, glucose and sorbitol. Exemplary such binders include polyvinylpyrrolidone, gelatin, starch, sucrose, methyl cellulose, ethyl cellulose, hydroxypropyl cellulose and hydroxypropyl alkylcellulose. Exemplary such lubricants include stearic acid, zinc stearate, magnesium stearate, calcium steatrate, talc, sodium benzoate, boric acid and colloidal silica.

The pharmaceutically acceptable additives may be used in an amount of 10 to 80 wt % based on the total weight of the formulation. When the amount of the additives is less than 10 wt %, the formulation may have low hardness, and when higher than 80 wt %, the sustained release effect may be compromised.

Also, the sustained release formulation of the present invention may further comprise a coating layer including coating agents, plasticizers, colorants, antioxidants, talc, titanium dioxide or flavors, in order to provide color, stability and a desired drug release pattern to the formulation. Exemplary such coating agents include ethyl cellulose, shellac, ammonio methacylate copolymer, hydroxyalkyl cellulose phthalate, hydroxyalkyl methylcellulose phthalate, cellulose acetate phthalate, sodium cellulose acetate phthalate, cellulose ester phthalate, cellulose ether phthalate, an anionic copolymer of methacylic acid and methyl or ethyl methacylate, polyvinylacetate, polyvinylpyrrolidone, polyvinylalcohol, hydroxymethyl cellulose, hydroxyethyl cellulose, hydroxypropyl cellulose, hydroxybutyl cellulose, hydroxypentyl cellulose, hydroxypropyl methylcellulose, hydroxypropyl butylcellulose, hydroxypropyl pentylcellulose, Opadry™ (Colorcon Co.), Acryl-EZE™ (Colorcon Co.), hydroxypropyl methylcellulose phthalate, hydroxypropyl methylcellulose acetate succinate and cellulose acetate phthalate, which may be used in an amount of 60 to 100 wt % based on the total weight of the coating layer. Exemplary such plasticizers include castor oil, fatty acids, triethylcitrate, substituted triglyceride, glyceride and polyethylene glycol having a molecular weight of 300 to 50,000, their derivatives, and a mixture thereof, which may be used in an amount of 0 to 40 wt % based on the total weight of the coating layer.

Furthermore, the present invention provides a method for preparing the inventive sustained release formulation in the form of a tablet, comprising the steps of (i) mixing an SSRI as an active ingredient, a water-soluble and erodible polymer, a hydrogel for shape sustenance, a binder and a diluent, and granulating the resulting mixture to produce granules; and (ii) adding a lubricant to the granules, followed by tableting the lubricated granules.

The inventive sustained release formulation in the form of a tablet may be prepared by an alternative method, comprising the steps of (i) mixing an SSRI as an active ingredient, a binder and a diluent, and granulating the resulting mixture to produce primary granules; (ii) adding a water-soluble and erodible polymer, a hydrogel for shape sustenance and a second diluent to the primary granules, and granulating the resulting mixture to produce secondary granules; and (iii) adding a lubricant to the secondary granules, followed by tableting the lubricated granules.

The method of the present invention may further comprise the step of coating the tablets thus prepared with coating agents.

In the present invention, the coating agent and the binder may be respectively used in the form of a solution in water or an organic solvent. Exemplary such organic solvents include methanol, ethanol, isopropanol, acetone, chloroform, dichloromethane and a mixture thereof.

The sustained release formulation of the present invention can release an SSIR having a relative low water-solubility at a constant rate following zero order kinetics by controlling the matrix erosion while preventing drug destruction caused, e.g., by gastrointestinal movements, thereby preventing side effects induced from the burst release of the drug into the blood.

The following Examples are intended to further illustrate the present invention without limiting its scope.

### EXAMPLES 1 TO 9

### Preparation of Matrix Tablet Comprising Paroxetine Hydrochloride

Paroxetine hydrochloride (solubility: 5.4 mg/ml), hydroxypropyl methylcellulose (HPMC) 2910 (methoxyl group: 28-30%, hydroxypropoxyl group: 7-12%, viscosity: 50 cps) as a water-soluble and erodible polymer, hydroxypropyl methylcellulose (HPMC) 2208 (methoxyl group: 19-24%, hydroxypropoxyl group: 4-12%, viscosity: 100 cps) as a hydrogel for shape sustenance, a mixture of microcrystalline cellulose and calcium hydrogen phosphate as a diluent and 5.4 g of polyvinylpyrrolidone dissolved in 80 g of ethanol as a binder were mixed according to the amounts shown in Table 1 and the resulting mixture was filtered through No. 14 mesh to obtain granules. The granules were dried and filtered through No. 18 mesh, to which a magnesium stearate lubricant was added. Then, the resulting mixture was compressed to obtain a tablet.

TABLE 1

| Ingredient | | | | | | | | | | |
|---|---|---|---|---|---|---|---|---|---|---|
| (wt %) | | Ex. 1 | Ex. 2 | Ex. 3 | Ex. 4 | Ex. 5 | Ex. 6 | Ex. 7 | Ex. 8 | Ex. 9 |
| Drug | Paroxetine | 7.9 | 7.9 | 7.9 | 7.9 | 7.9 | 7.9 | 7.9 | 7.9 | 7.9 |
| | hydrochloride | | | | | | | | | |
| Erodible | HPMC 2910 | 31.7 | 38.8 | 31.7 | 31.7 | 33.48 | 32.6 | 36.1 | 32.6 | 32.6 |
| polymer | | | | | | | | | | |
| Hydrogel | HPMC 2208 | 31.7 | 31.7 | 38.8 | 31.7 | 33.48 | 32.6 | 32.6 | 36.1 | 32.6 |
| for shape | | | | | | | | | | |
| sustenance | | | | | | | | | | |
| Diluent | Microcrystalline | 8.8 | 8.8 | 8.8 | 15.9 | 10.57 | 9.7 | 9.7 | 9.7 | 13.2 |
| | cellulose | | | | | | | | | |
| | Calcium | 15.9 | 8.8 | 8.8 | 8.8 | 10.57 | 13.2 | 9.7 | 9.7 | 9.7 |
| | hydrogen | | | | | | | | | |
| | phosphate | | | | | | | | | |
| Binder | Polyvinyl- | 3.0 | 3.0 | 3.0 | 3.0 | 3.0 | 3.0 | 3.0 | 3.0 | 3.0 |
| | pyrrolidone | | | | | | | | | |
| Lubricant | Magnesium | 1.0 | 1.0 | 1.0 | 1.0 | 1.0 | 1.0 | 1.0 | 1.0 | 1.0 |
| | stearate | | | | | | | | | |

### TEST EXAMPLE 1

The tablets prepared in Examples 1 to 9 were subjected to in vitro release-tests using a USP apparatus II (paddle method). The release rate of paroxetin hydrochloride from each of the tablets was measured under the conditions of: an artificial intestinal solution (Solution II, pH 6.8), paddle type, 900 ml release solution and 50 rpm rotational speed for 12 hours. The result is shown in Table 2.

TABLE 2

| Time | | | | | | | | | |
|---|---|---|---|---|---|---|---|---|---|
| (hr) | Ex. 1 | Ex. 2 | Ex. 3 | Ex. 4 | Ex. 5 | Ex. 6 | Ex. 7 | Ex. 8 | Ex. 9 |
| 0 | 0 | 0 | 0 | 0 | 0 | 0 | 0 | 0 | 0 |
| 1 | 13.5 | 10.7 | 10.5 | 10.9 | 8.5 | 8.9 | 13.3 | 10.7 | 10.9 |
| 3 | 27.0 | 24.1 | 21.5 | 22.8 | 22.8 | 24.3 | 23.8 | 23.3 | 23.6 |
| 5 | 41.7 | 37.3 | 31.5 | 34.5 | 34.7 | 38.0 | 35.5 | 34.7 | 35.2 |
| 7 | 56.8 | 50.2 | 41.2 | 46.6 | 46.4 | 51.4 | 47.4 | 45.3 | 46.4 |
| 12 | 90.0 | 79.7 | 71.3 | 77.8 | 73.8 | 81.1 | 75.1 | 70.6 | 72.2 |

As can be seen from Table 2, the release rate is primarily affected by the amount of the hydrogel; it becomes slow as the amount of the hydrogel increases. Also, the time-dependent change in the in vitro release pattern follows zero order kinetics having a correlation coefficient of 0.8 to 1.0.

### COMPARATIVE EXAMPLES 1 AND 2 AND EXAMPLES 10 TO 13

### Preparation of Matrix Tablet Comprising Paroxetine Hydrochloride

Tablets having the compositions listed in Table 3 were each prepared by repeating the procedure of Examples 1 to 9.

TABLE 3

| Ingredient | | | | | | | |
|---|---|---|---|---|---|---|---|
| (wt %) | | Com. Ex. 1 | Com. Ex. 2 | Ex. 10 | Ex. 11 | Ex. 12 | Ex. 13 |
| Drug | Paroxetine | 7.9 | 7.9 | 7.9 | 7.9 | 7.9 | 7.9 |
| | hydrochloride | | | | | | |
| Erodible | HPMC 2910 | 33.3 | — | 34.1 | 30.0 | 40.0 | 35.0 |
| polymer | | | | | | | |
| Hydrogel | HPMC 2208 | — | 29.4 | 31.7 | — | — | — |
| for shape | Xanthan gum | — | — | — | 10.0 | — | — |
| sustenance | Sodium alginate | — | — | — | — | 20.0 | — |
| | Hydroxypropyl | — | — | — | — | — | 20.0 |
| | methylcellulose | | | | | | |
| | acetyl succinate | | | | | | |
| Diluent | Microcrystalline | 27.2 | 29.1 | 11.2 | 24.0 | 12.5 | 16.5 |
| | cellulose | | | | | | |
| | Calcium | 27.4 | 29.4 | 11.2 | 24.1 | 15.6 | 16.6 |
| | hydrogen | | | | | | |
| | phosphate | | | | | | |
| Binder | Polyvinyl- | 3.2 | 3.2 | 3.0 | 3.0 | 3.0 | 3.0 |
| | pyrrolidone | | | | | | |
| Lubricant | Magnesium | 1.0 | 1.0 | 1.0 | 1.0 | 1.0 | 1.0 |
| | stearate | | | | | | |

### TEST EXAMPLE 2

In vitro release-tests were conducted for the tablets prepared in Comparative Examples 1 and 2 and Examples 10 and 11 by repeating the method of Test Example 1, except for varying the rotational speed from 50 rpm, to 75 rpm and to 100 rpm over a period of 12 hours. The measured release rates are shown in Table 4.

TABLE 4

| | Com. Ex. 1 | | | Com. Ex. 2 | | | Ex. 10 | | | Ex. 11 | | |
|---|---|---|---|---|---|---|---|---|---|---|---|---|
| Time | 50 | 75 | 100 | 50 | 75 | 100 | 50 | 75 | 100 | 50 | 75 | 100 |
| (hr) | rpm | rpm | rpm | rpm | rpm | rpm | rpm | rpm | rpm | rpm | rpm | rpm |
| 0 | 0.0 | 0.0 | 0.0 | 0.0 | 0.0 | 0.0 | 0.0 | 0.0 | 0.0 | 0.0 | 0.0 | 0.0 |
| 1 | 16.9 | 22.5 | 28.3 | 17.0 | 21.2 | 22.4 | 10.3 | 12.1 | 12.7 | 13.8 | 15.6 | 16.9 |
| 2 | 27.7 | 39.3 | 47.6 | 27.7 | 32.6 | 36.1 | 15.7 | 18.3 | 22.9 | 22.0 | 24.5 | 27.5 |
| 3 | 41.3 | 57.9 | 70.4 | 36.9 | 41.8 | 48.2 | 21.6 | 26.7 | 32.5 | 32.1 | 37.2 | 43.8 |
| 4 | 51.4 | 73.6 | 92.6 | 46.1 | 52.6 | 62.5 | 27.2 | 32.8 | 39.6 | 42.1 | 48.7 | 55.2 |
| 5 | 68.1 | 89.1 | 98.2 | 55.9 | 63.7 | 74.1 | 32.9 | 39.0 | 46.7 | 51.8 | 55.9 | 62.3 |
| 6 | 81.4 | 101.4 | — | 64.7 | 75.0 | 86.9 | 40.0 | 44.6 | 51.3 | 61.2 | 67.5 | 75.5 |
| 7 | 96.6 | — | — | 73.2 | 84.1 | 99.7 | 45.2 | 50.7 | 58.1 | 70.1 | 77.8 | 83.2 |
| 8 | — | — | — | 80.3 | 91.3 | — | 51.8 | 57.4 | 63.7 | 79.0 | 85 | 91.3 |
| 10 | — | — | — | 92.6 | 98.5 | — | 60.9 | 65.6 | 75.6 | 92.8 | 98.6 | — |
| 12 | — | — | — | 100.2 | — | — | 70.7 | 75.4 | 84.2 | 100.5 | 99.3 | — |

As can be seen from Table 4, the released amounts of the drug by the tablets of Comparative Examples 1 and 2 increased by 30 to 40% as the rotational speed increases, while the tablets of Examples 10 and 11 comprising a water-soluble and erodible polymer together with a hydrogel for shape sustenance exhibited a relatively steady release rates even when the rotation rate was varied. Thus, the inventive tablets are little affected by physical factors such as gastrointestinal movements.

### TEST EXAMPLE 3

In vitro release-tests were conducted for the tablets prepared in Examples 12 and 13 by repeating the method of Test Example 1, except for using an artificial gastric solution (Solution I, pH 1.2) for 2 hours and then an artificial intestinal solution (Solution II, pH 6.8) for 7 hours. The measured released amounts of the drug are shown in Table 5.

TABLE 5

| Time | | |
|---|---|---|
| (hr) | Ex. 12 | Ex. 13 |
| 0 | 0.0 | 0.0 |
| 0.5 | 1.1 | 0.0 |
| 1 | 3.0 | 0.0 |
| 1.5 | 3.5 | 1.2 |
| 2 | 4.0 | 5.0 |
| 3 | 18.5 | 12.4 |
| 4 | 32.6 | 36.6 |
| 5 | 49.7 | 54.7 |
| 6 | 62.3 | 71.5 |
| 7 | 78.1 | 86.1 |
| 8 | 92.4 | 95.3 |
| 9 | 101.6 | 99.8 |

As can be seen from Table 5, the tablets of Examples 12 and 13 exhibit very steady release patterns in the stomach and the upper small intestine, as well as in the small intestine without instant burst release of the drug, which results from the use of a hydrogel having pH-dependent, enteric coating properties of the inventive tablets.

### EXAMPLE 14

### Coating of Matrix Tablet

The tablet prepared in Example 10 was successively spray-coated with a mixture of hydroxypropyl methylcellulose 2910 and triethyl citrate, and Acryl-EZE (Colorcon Co.) which is an enteric coating agent containing a colorant, in amounts shown in Table 6.

TABLE 6

| | Ingredient | Amount (mg) |
|---|---|---|
| Coating I | Hydroxypropyl | 5.40 |
| | methylcellulose 2910 | |
| | Triethyl citrate | 0.54 |
| Coating II | Acryl-EZE | 12.60 |

### TEST EXAMPLE 4

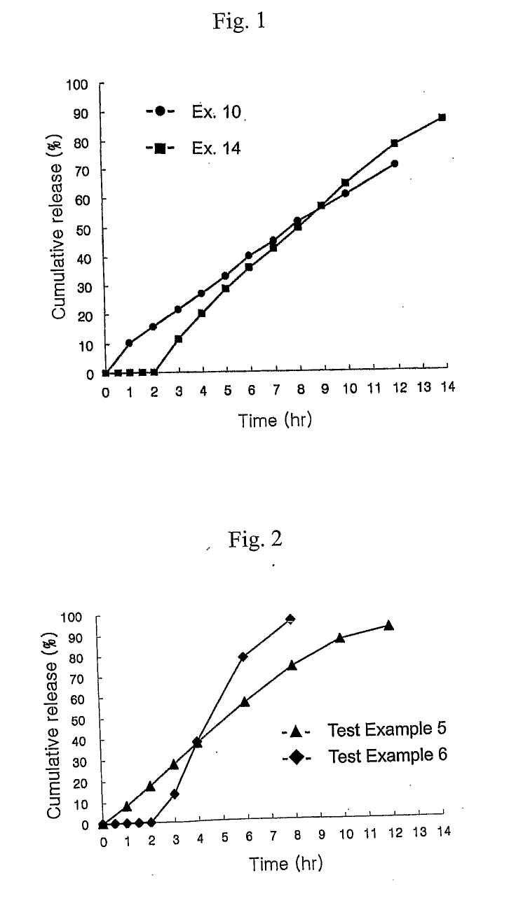

The coated tablet prepared in Example 14 was subjected to in vitro release-test using a USP apparatus I (basket method). The drug release rate was measured under the conditions of: an artificial gastric solution (Solution I, pH 1.2) for 2 hours and then an artificial intestinal solution (Solution II, pH 6.8), basket type, 900 ml release solution and 100 rpm rotational speed for 12 hours. The result is shown in Table 7, and the release profiles for the tablets of Examples 10 and 14 are shown in FIG. 1.

TABLE 7

| Time | |
|---|---|
| (hr) | Ex. 14 |
| 0 | 0.0 |
| 0.5 | 0.0 |
| 1 | 0.0 |
| 1.5 | 0.0 |
| 2 | 0.0 |
| 3 | 11.2 |
| 4 | 20.3 |
| 5 | 28.5 |
| 6 | 35.9 |
| 7 | 42.6 |
| 8 | 49.5 |
| 9 | 56.8 |
| 10 | 64.6 |
| 12 | 77.9 |
| 14 | 86.8 |

As can seen from Table 7, the tablet of Example 14 exhibits a steady release pattern in only the artificial intestinal solution without any release in the artificial gastric solution. This result shows that the active drug (paroxetine) can be selectively delivered to the small intestinal.

### EXAMPLES 15 TO 20

### Preparation of Coated Matrix Tablet Comprising Paroxetine Hydrochloride

The tablets having a core part and coating parts I and II having compositions listed in Table 8 were prepared by repeating the procedure of Examples 1 to 9 for forming the core part and the procedure of Example 8 for forming the coating part.

TABLE 8

| | Ingredient | | | | | | |
|---|---|---|---|---|---|---|---|
| | (mg) | Ex. 15 | Ex. 16 | Ex. 17 | Ex. 18 | Ex. 19 | Ex. 20 |
| Core | Paroxetine | 14.22 | 14.22 | 14.22 | 14.22 | 14.22 | 14.22 |
| | hydrochloride | | | | | | |
| | HPMC 2910 | 51.00 | 51.00 | 76.50 | 93.50 | 51.00 | 64.60 |
| | HPMC 2208 | 8.50 | 8.50 | 8.50 | 8.50 | 8.50 | 8.50 |
| | Microcrystalline | 15.86 | 15.86 | 15.86 | 15.86 | 15.86 | 15.86 |
| | cellulose | | | | | | |
| | Calcium hydrogen | 68.52 | 68.52 | 43.02 | 26.02 | 68.52 | 54.92 |
| | phosphate | | | | | | |
| | Polyvinyl- | 10.20 | 10.20 | 10.20 | 10.20 | 10.20 | 10.20 |
| | pyrrolidone | | | | | | |
| | Magnesium stearate | 1.70 | 1.70 | 1.70 | 1.70 | 1.70 | 1.70 |
| | Total core weight | 170.00 | 170.00 | 170.00 | 170.00 | 170.00 | 170.00 |
| Coating I | Talc | — | 6.79 | 6.79 | 6.79 | — | — |
| | Sucrose | — | 3.83 | 3.83 | 3.83 | — | — |
| | HPMC 2910 | — | 1.28 | 1.28 | 1.28 | 7.14 | 7.14 |
| | Ethyl cellulose | 4.36 | — | — | — | 3.06 | 3.06 |
| | Triethyl citrate | 0.39 | — | — | — | 0.85 | 0.85 |
| Coating II | Acryl-EZE | 8.50 | — | — | — | — | — |
| | Hydroxypropyl | — | 8.50 | 10.20 | 11.90 | 11.90 | 11.90 |
| | methylcellulose | | | | | | |
| | phthalate | | | | | | |
| | Triethyl citrate | — | 0.68 | 0.82 | 0.95 | 0.95 | 0.95 |

### TEST EXAMPLE 5

The coated tablets prepared in Examples 15 to 20 were subjected to in vitro release-tests using a USP apparatus II (paddle method). The drug release rate was measured under the conditions of: an artificial intestinal solution (Solution II, pH 6.8), paddle type, 900 ml release solution and 50 rpm rotational speed for 12 hours. The result is shown in Table 9.

TABLE 9

| Time | | | | | | |
|---|---|---|---|---|---|---|
| (hr) | Ex. 15 | Ex. 16 | Ex. 17 | Ex. 18 | Ex. 19 | Ex. 20 |
| 0 | 0.0 | 0.0 | 0.0 | 0.0 | 0.0 | 0.0 |
| 1 | 4.3 | 11.1 | 9.3 | 8.8 | 7.1 | 8.3 |
| 2 | 12.4 | 22.5 | 19.1 | 20.6 | 16.8 | 17.5 |
| 3 | 20.9 | 34.5 | 29.3 | 31.6 | 26.4 | 27.4 |
| 4 | 30.5 | 46.4 | 39.7 | 42.4 | 36.2 | 37.2 |
| 6 | 48.3 | 68.2 | 59.3 | 60.5 | 55.5 | 56.0 |
| 8 | 64.7 | 85.4 | 75.4 | 76.0 | 73.6 | 73.2 |
| 10 | 78.1 | 92.9 | 87.8 | 87.0 | 86.6 | 86.3 |
| 12 | 84.8 | 94.6 | 93.5 | 92.6 | 91.6 | 91.8 |

### TEST EXAMPLE 6

The coated tablets prepared in Examples 15 to 20 were subjected to in vitro release-tests using a USP apparatus II (paddle method). The drug release rate was measured under the conditions of: (i) 0.1 mol/l hydrochloric acid solution, paddle type, 750 ml release solution and 150 rpm rotation time (Solution I, pH 1.2) for 2 hours and (ii) Tris buffer solution (pH 7.5), paddle type, 1000 ml release solution and 150 rpm rotational speed for 6 hours. The result is shown in Table 10, and the release profiles for the tablet of Example 20 according to Test Examples 5 (-▴-) and 6 (-♦-) are shown in FIG. 2.

TABLE 10

| Time | | | | | | |
|---|---|---|---|---|---|---|
| (hr) | Ex. 15 | Ex. 16 | Ex. 17 | Ex. 18 | Ex. 19 | Ex. 20 |
| 0 | 0.0 | 0.0 | 0.0 | 0.0 | 0.0 | 0.0 |
| 0.5 | 0.0 | 0.0 | 0.0 | 0.0 | 0.0 | 0.0 |
| 1 | 0.0 | 0.0 | 0.0 | 0.0 | 0.0 | 0.0 |
| 1.5 | 0.0 | 0.0 | 0.0 | 0.0 | 0.0 | 0.0 |
| 2 | 0.0 | 0.0 | 0.0 | 0.0 | 0.0 | 0.0 |
| 3 | 8.3 | 16.3 | 16.2 | 11.2 | 12.1 | 13.4 |
| 4 | 23.1 | 36.4 | 37.9 | 32.9 | 38.2 | 37.7 |
| 6 | 64.7 | 77.6 | 75.7 | 65.9 | 83.0 | 78.0 |
| 8 | 92.0 | 94.1 | 95.3 | 88.5 | 95.8 | 95.8 |

As can seen from Tables 9 and 10, the inventive tablets of Examples 15 to 20 can repress or sustain the initial burst release of the drug from the core part since the first coating layer blocks the reaction between the core part and the second enteric coating layer, thereby protecting the enteric layers.

While the invention has been described with respect to the above specific embodiments, it should be recognized that various modifications and changes may be made and also fall within the scope of the invention as defined by the claims that follow.

What is claimed is:

1. A sustained release formulation of a selective serotonin reuptake inhibitor (SSRI), comprising the SSRI as an active ingredient and a matrix comprising a water-soluble and erodible polymer, a hydrogel for shape sustenance and a pharmaceutically acceptable additive, wherein the SSRI is embedded in the matrix.

2. The formulation of claim 1, wherein the release rate of the SSRI follows zero order kinetics with a correlation coefficient ranging from 0.8 to 1.0.

3. The formulation of claim 1, which comprises 0.5 to 80 wt % of the SSRI, 5 to 40 wt % of the polymer, 5 to 40 wt % of the hydrogel and 10 to 80 wt % of the pharmaceutically acceptable additive, based on the total weight of the formulation.

4. The formulation of claim 1, wherein the weight ratio of polymer hydrogel ranges from 1:0.1 to 8.

5. The formulation of claim 1, wherein the SSRI is selected from the group consisting of paroxetine, sertraline, fluoxetine, fluvoxamine and pharmaceutically acceptable salts thereof.

6. The formulation of claim 1, wherein the water-soluble and erodible polymer has a pH-independent viscosity of 3 to 400 centipoise (cps).

7. The formulation of claim 6, wherein the polymer is selected from the group consisting of hydroxyalkyl cellulose, hydroxypropyl alkylcellulose, polyethylene oxide, propylene glycol alginate, polyvinylpyrrolidone, polyvinylalcohol, sodium carboxymethyl cellulose and a mixture thereof.

8. The formulation of claim 7, wherein the hydroxyalkyl cellulose is hydroxyethyl cellulose and the hydroxypropyl alkylcellulose is hydroxypropyl methylcellulose having a methoxyl group content of 27 to 30% and a hydroxyproproxyl group content of 4 to 12%.

9. The formulation of claim 1, wherein the hydrogel for shape sustenance is a water-insoluble polymer, a water-soluble polymer having a solubility lower than that of the water-soluble, erodible polymer and a viscosity of 100 to 200,000 cps, or an enteric polymer having a pH-dependent solubility.

10. The formulation of claim 9, wherein the hydrogel is selected from the group consisting of hydroxyalkyl cellulose, hydroxypropyl alkylcellulose, sodium alginate, xanthan gum, locust bean gum, cellulose gum, ethyl cellulose, ammonio methacylate copolymer, anionic copolymer of methacrylic acid and methyl or ethyl methacylate, hydroxypropyl methylcellulose acetate succinate, hydroxypropyl methylcellulose phthalate, carbopol and a mixture thereof.

11. The formulation of claim 10, wherein the hydroxyalkyl cellulose is hydroxypropyl cellulose and the hydroxypropyl alkylcellulose is hydroxypropyl methylcellulose having a methoxyl group content of 19 to 24% and a hydroxyproproxyl group content of 4 to 12%.

12. The formulation of claim 1, wherein the pharmaceutically acceptable additive is selected from the group consisting of diluents, binders, lubricants and a mixture thereof.

13. The formulation of claim 1, which further comprises a coating layer containing a coating agent.

14. A method for preparing the formulation of claim 1 in the form of a tablet, comprising the steps of (i) mixing the SSRI, the water-soluble and erodible polymer, the hydrogel, a binder and a diluent, and granulating the resulting mixture to produce granules; and (ii) adding a lubricant to the granules, followed by tableting the lubricated granules.

15. A method for preparing the formulation of claim 1 in the form of a tablet, comprising the steps of (i) mixing the SSRI, a binder and a diluent, and granulating the resulting mixture to produce primary granules; (ii) adding the water-soluble and erodible polymer, the hydrogel and a second diluent to the primary granules, and granulating the resulting mixture to produce secondary granules; and (iii) adding a lubricant to the secondary granules, followed by tableting the lubricated granules.

16. The method of claim 14 or 15, which further comprises the step of coating the tablets with a coating agent.
